(19) United States
(12) Patent Application Publication
(10) Pub. No.: US 2023/0404364 A1
(43) Pub. Date: Dec. 21, 2023

(54) STENT DELIVERY SYSTEM AND STENT DELIVERY METHOD

(71) Applicant: OLYMPUS CORPORATION, Tokyo (JP)

(72) Inventors: Yuji SAKAMOTO, Kunitachi-shi (JP); Shintaro INOUE, Westborough, MA (US); Anthony PIROZZI, Raleigh, NC (US); Gloria YEE, Wellesley, MA (US); Brandon RANALLI, Quincy, MA (US)

(73) Assignee: OLYMPUS CORPORATION, Tokyo (JP)

(21) Appl. No.: 18/200,163

(22) Filed: May 22, 2023

Related U.S. Application Data
(60) Provisional application No. 63/344,740, filed on May 23, 2022.

(51) Int. Cl. A61B 1/00; A61B 1/018; A61F 2/966

(52) U.S. Cl. CPC A61B 1/000094; A61B 1/018; A61F 2/966; A61B 1/0016; A61B 1/00006

(57) ABSTRACT

A stent delivery system includes: a stent delivery device configured to carry a stent to a stenosis and indwell the stent; an observation device configured to observe the stenosis; and one or more processors comprising hardware, the one or more processors being configured to: acquire an observation image from the observation device, and determine, based on the observation image, at least one insertion position, which is a position within the stenosis where the stent delivery device is recommended to be inserted.

### CROSS-REFERENCE TO RELATED APPLICATIONS

The present application claims priority based on U.S. Patent Provisional Application No. 63/344,740 provisionally filed in the United States on May 23, 2022, the contents of which is incorporated herein by reference.

### BACKGROUND

The present disclosure relates to stent delivery systems and stent delivery methods.

A stent delivery device is used when a self-expanding stent is placed in a stenosis or occlusion (hereinafter referred to as “stenosis, etc.”) that occurs in a lumen. In a conventional stent delivery device, a stent is housed in a gap between an inner sheath and an outer sheath, and the stent is exposed and expanded by retracting the outer sheath with respect to the inner sheath. The stent is left in the lumen by removing the inner sheath from the stent.

US Patent Publication No. 2017/0086929 discloses an example in which a robotic catheter system that performs a procedure by remotely operating a catheter system is applied to ERCP (endoscopic retrograde cholangiopancreatography).

### SUMMARY

A stent delivery system according to a first aspect of the present disclosure includes: a stent delivery device configured to carry a stent to a stenosis and indwell the stent; an observation device configured to observe the stenosis; and one or more processors comprising hardware, the one or more processors being configured to: acquire an observation image from the observation device, and determine, based on the observation image, at least one insertion position, which is a position within the stenosis where the stent delivery device is recommended to be inserted.

A stent delivery method for inserting a stent delivery device to a stenosis position according to a second aspect of the present disclosure incudes: obtaining an observation image from an observation device used to observe a stenosis; determining at least one insertion position, which is a position in a stenosis where the stent delivery device is recommended to be inserted, based on the observation image.

A control device for a stent delivery system according to a third aspect includes a stent delivery device that carries and indwells a stent to a stenosis, an observation device used to observe the stenosis, and one or more processors comprising hardware, wherein the one or more processors being configured to determine at least one insertion position, which is a position within the stenosis where the stent delivery system is recommended to be inserted, based on the observation image acquired from the observation device.

### BRIEF DESCRIPTION OF THE DRAWINGS

FIG. 1 is a diagram showing the overall configuration of an endoscope system according to a first embodiment.

FIG. 2 is a view showing an insertion portion of an endoscope of the endoscope system.

FIG. 3 is a perspective view of a connecting portion of the endoscope.

FIG. 4 is a diagram showing a stent delivery device of the endoscope system.

FIG. 5 is a functional block diagram of a driving device of the endoscope system.

FIG. 6 is a diagram showing a delivery device driving device of the driving device.

FIG. 7 is a diagram showing a connecting portion driving device of the driving device.

FIG. 8 is a functional block diagram of a video control device of the endoscope system.

FIG. 9 is a diagram showing a bile duct in which a stent is placed.

FIG. 10 is a flow chart showing the steps of the procedure.

FIG. 11 is a diagram showing the stent delivery device moved to the vicinity of the stenosis.

FIG. 12 is a control flowchart of the main controller of the control device in the endoscope system in the stent placement process.

FIG. 13 is an X-ray image acquired by an observation image acquisition step.

FIG. 14 is a view showing the stent delivery device moved to the first intermediate insertion position.

FIG. 15 is a view showing the stent delivery device moved to a second halfway insertion position.

FIG. 16 is a diagram showing a second input device of an endoscope system according to a second embodiment.

### DETAILED DESCRIPTION

### First Embodiment

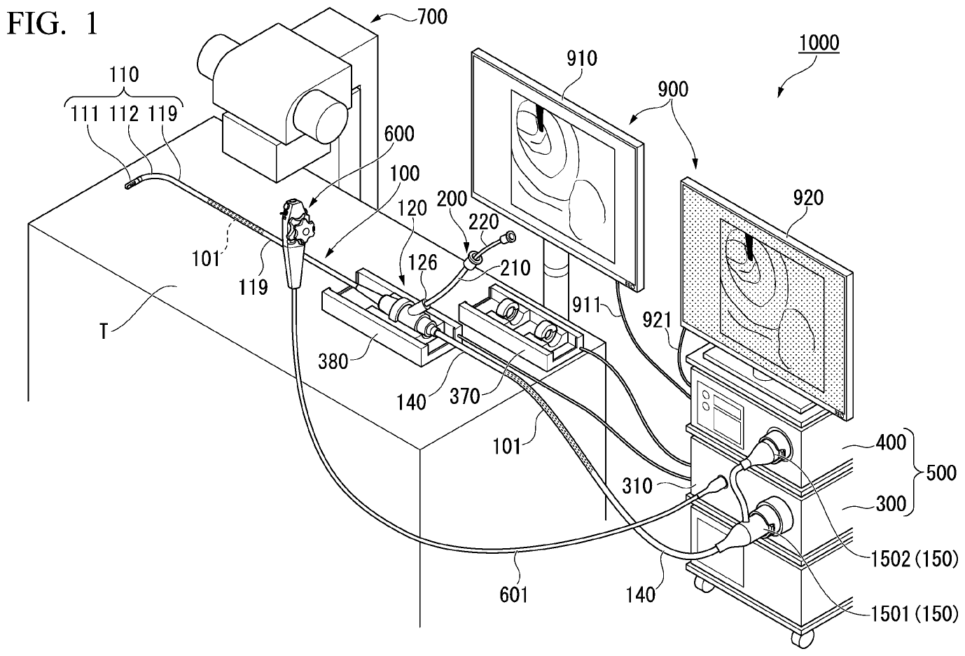

An endoscope system 1000 according to the first embodiment of the present disclosure will be described with reference to FIGS. 1 to 15. FIG. 1 is a diagram showing the overall configuration of the endoscope system 1000.

### [Endoscope System 1000]

The endoscope system (stent delivery system) 1000 is a medical system for observing and treating the inside of a patient lying on an operating table T. The endoscope system 1000 includes an endoscope 100, a stent delivery device 200, a drive device 300 (driving actuator), a video control device 400, an operation device 600, an observation device 700, and a display device 900.

The endoscope 100 is a device that is inserted into a patient's lumen to observe and treat an affected area. The endoscope 100 is detachable from the driving device 300 and the video control device 400. An internal path 101 is formed inside the endoscope 100. In the following description, in the endoscope 100, the side that is inserted into the patient's lumen is referred to as the “distal side A1”, and the side that is attached to the driving device 300 is referred to as the “proximal end side A2”.

As shown in FIG. 1, the stent delivery device 200 is an indwelling device that is inserted into the internal path 101 of the endoscope 10 through a forceps opening 126 provided in a connecting portion 120 of the endoscope 100, and delivers a stent 230 into the lumen of the patient.

The driving device 300 is detachably connected to the endoscope 100 and the operation device 600. The driving device 300 drives a built-in motor to electrically drive the endoscope 100 based on an operation input to the operation device 600. Further, the driving device 300 drives a built-in pump or the like based on an operation input to the operation device 600 to cause the endoscope 100 to perform air supply and suction.

The driving device 300 includes a delivery device driving device 370 detachably connected to an operating portion 240 of the stent delivery device 200, and a connecting portion driving device 380 detachably connected to the connecting portion 120 of the endoscope 100.

The video control device 400 is detachably connected to the endoscope 100 and acquires captured images from the endoscope 100. The video control device 400 causes the display device 900 to display captured images acquired from the endoscope 100 and GUI images and CG images to provide information to the operator.

The drive device 300 and the video control device 400 constitute a control device 500 that controls the endoscope system 1000. The controller 500 may further include peripherals such as a video printer. The driving device 300 and the video control device 400 may be an integrated device.

The operation device 600 is detachably connected to the driving device 300 via an operation cable 601. The operation device 600 may be capable of communicating with the driving device 300 by wireless communication instead of wired communication. The operator can electrically drive the endoscope 100 by operating the operation device 600.

The observation device 700 is a known X-ray fluoroscopy device that emits X-rays from outside the body to observe a patient. The observation device 700 may include a device for observing a patient by CT (Computed Tomography) or MRI (Magnetic Resonance Imaging). The observation device 700 is connected to the video control device 400 via a connection cable (not shown). Note that the observation device 700 is not limited to the device shown in FIG. 1.

The display device 900 includes an endoscope image display device 910 and an observation image device 920. The endoscope image display device 910 is a device capable of displaying images such as an LCD. The endoscope image display device 910 is connected to the video control device 400 via a display cable 911.

The observation image device 920 is a device capable of displaying an X-ray image. The observation image device 920 is connected to the video control device 400 via a display cable 921. When the observation device 700 includes a device for observing a patient by CT or MRI, the observation image device 920 includes a device capable of displaying CT images and MRI images.

Next, each device of the endoscope system 1000 will be described in detail.

### [Endoscope 100]

The endoscope 100 is a flexible endoscope that is side-viewing, and includes an insertion portion 110, a connecting portion 120, an extracorporeal flexible portion 140, a detachable portion 150, a bending wire 160, and an internal object 170.

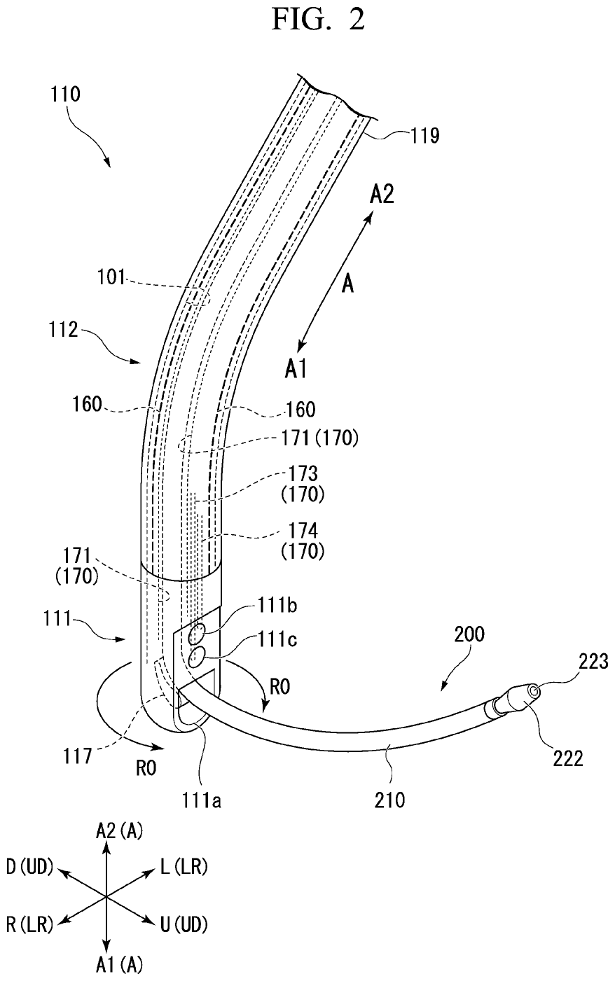

FIG. 2 is a diagram showing the insertion portion 110 of the endoscope 100.

The internal path 101 extending along the longitudinal direction A of the endoscope 100 from the distal end of the insertion portion 110 to the proximal end is formed inside the endoscope 100. The bending wire 160 and the internal object 170 are inserted into the internal path 101. The bending wire 160 has an operation wire for bending a bending portion 112 and an elevator operation wire for driving an elevator 117. The internal object 170 has a channel tube 171, an imaging cable 173, and a light guide 174.

### [Insertion Portion 110]

The insertion portion 110 is an elongated long member that can be inserted into a lumen. The insertion portion 110 has a distal end portion 111, a bending portion 112, and an internal soft portion 119. The distal end portion 111, the bending portion 112, and the internal soft portion 119 are connected in order from the distal end side.

The distal end portion 111 is formed of metal or the like into a substantially cylindrical shape. The distal end portion 111 has an opening portion 111a, an illumination portion 111b, and an imaging portion 111c. The opening portion 111a is provided on the side surface of the distal end portion 111 and communicates with the channel tube 171. As shown in FIG. 2, the stent delivery device 200 passing through the channel tube 171 protrudes from the opening portion 111a.

The illumination portion 111b is connected to a light guide 174 that guides illumination light, and emits illumination light that illuminates the imaging target. The imaging portion 111c includes an imaging element such as a CMOS, and images an object to be imaged. The imaging signal is sent to the video control device 400 via the imaging cable 173.

A riser 117 is provided near the opening portion 111a of the distal end portion 111. A proximal end portion of the raising base 117 is rotatably supported by the distal end portion 111. An elevator operating wire fixed to the distal end of the elevator 117 extends to the attachment/detachment portion 150 through the internal path 101.

The bending portion 112 can be bent in a vertical direction perpendicular to the longitudinal direction A (also referred to as a “UD direction”) or a horizontal direction perpendicular to the longitudinal direction A and the UD direction (also referred to as an “LR direction”). The distal end of the operation wire is fixed to the distal end side of the bending portion 112. The operating wire extends through the internal path 101 to the detachable portion 150.

The internal soft portion 119 is an elongated flexible tubular member. A bending wire 160, a channel tube 171, an imaging cable 173, and a light guide 174 are inserted through the internal path 101 formed in the internal soft portion 119.

### [Connecting Portion 120]

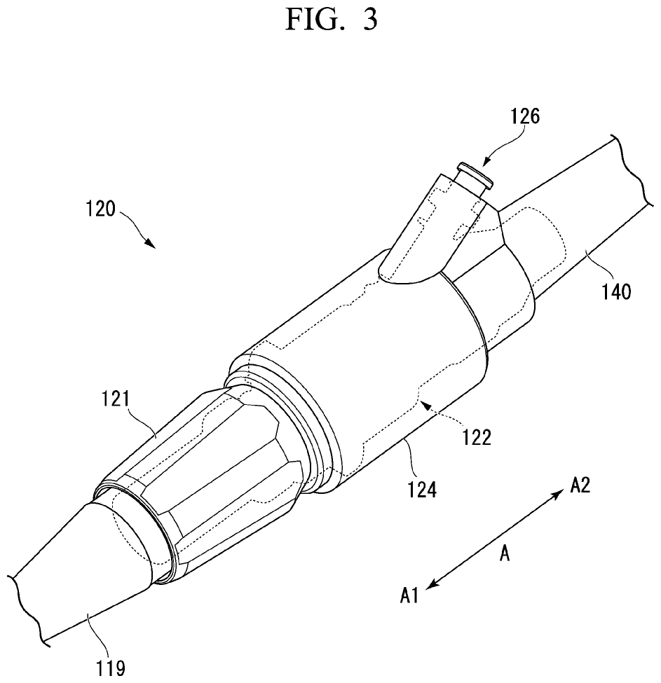

FIG. 3 is a perspective view of the connecting portion 120.

The connecting portion 120 is a member that connects the internal soft portion 119 and the extracorporeal soft portion 140 of the insertion portion 110. The connecting portion 120 includes a cylindrical member 121, a connecting portion main body 122, a bearing portion 124, and a forceps opening 126.

The cylindrical member 121 is formed in a cylindrical shape. The internal space of the cylindrical member 121 communicates with the internal space of the internal flexible portion 119 and forms part of the internal path 101. A bending wire 160, a channel tube 171, an imaging cable 173, and a light guide 174 are inserted through the inner space of the cylindrical member 121.

The connecting portion main body 122 is formed in a substantially cylindrical shape. A cylindrical member 121 is inserted into the distal end opening of the connecting portion main body 122. The internal space of the connecting portion main body 122 communicates with the internal space of the extracorporeal soft portion 140 and forms part of the internal path 101.

The bearing portion 124 connects the connecting portion main body 122 and the cylindrical member 121 so as to be rotatable around a rotation axis extending in the longitudinal direction A. Specifically, the bearing portion 124 is fixed to the connecting portion main body 122. The bearing portion 124 supports the cylindrical member 121 so as to be rotatable around a rotation axis extending in the longitudinal direction A.

The internal soft portion 119 is fixed to the cylindrical member 121. Therefore, the internal flexible portion 119 and the cylindrical member 121 rotate together with respect to the connecting portion main body 122.

The forceps opening 126 is an insertion port for inserting a treatment tool such as the stent delivery device 200. The forceps opening 126 is formed in a cylindrical shape.

The internal soft portion 119 and the external soft portion 140 are connected by a connecting portion 120 so as to be rotatable around a rotation axis extending in the longitudinal direction A. Therefore, when the operator rotates the internal soft portion 119 of the insertion portion 110 around the rotation axis extending in the longitudinal direction A, only the internal soft portion 119 can be rotated without rotating the extracorporeal soft portion 140 extending to the vicinity of the driving device 300.

When the operator rotates the internal soft portion 119 of the insertion portion 110 around the rotation axis extending in the longitudinal direction A, the forceps opening 126 attached to the connecting portion main body 122, which is a portion that does not rotate in conjunction with the internal soft portion 119, does not rotate. Since the position of the forceps opening 126 into which the treatment tool such as the stent delivery device 200 is inserted does not change, the operator can easily operate the treatment tool.

### [Extracorporeal Soft Portion 140]

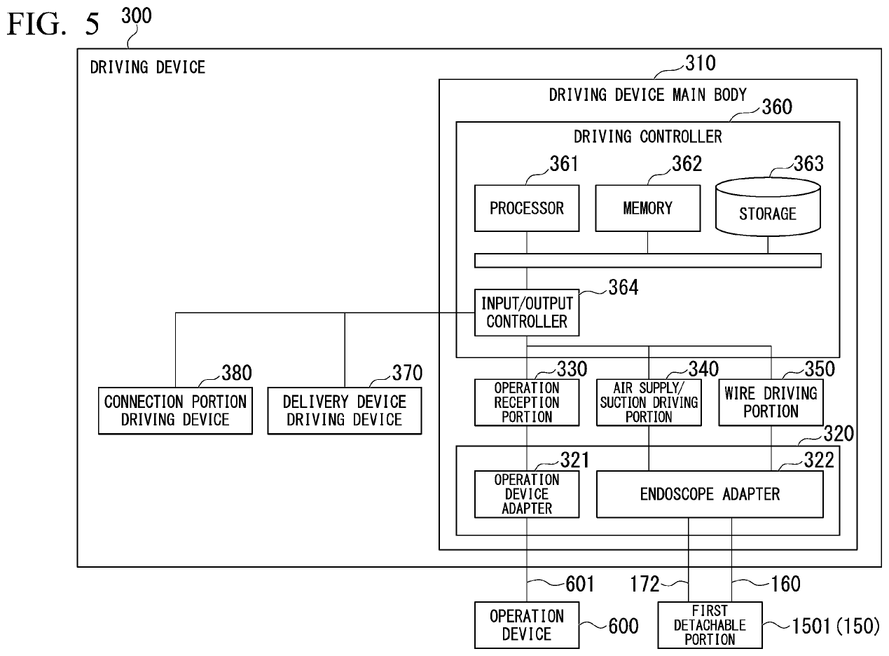

The extracorporeal soft portion 140 is a long tubular member. A bending wire 160, an imaging cable 173, a light guide 174, and an air supply/suction tube 172 (see FIG. 5) are inserted through the internal path 101 formed inside the extracorporeal soft portion 140. The air supply/suction tube 172 is connected to the channel tube 171.

### [Detachable Portion 150]

The detachable portion 150 includes a first detachable portion 1501 attached to the driving device 300 and a second detachable portion 1502 attached to the video control device 400, as shown in FIG. 1. Note that the first detachable portion 1501 and the second detachable portion 1502 may be an integral detachable portion.

The internal path 101 formed inside the extracorporeal soft portion 140 branches into a first detachable portion 1501 and a second detachable portion 1502. The bending wire 160 and the air supply/suction tube 172 are inserted through the first detachable portion 1501. The imaging cable 173 and the light guide 174 are inserted through the second detachable portion 1502.

### [Stent Delivery Device 200]

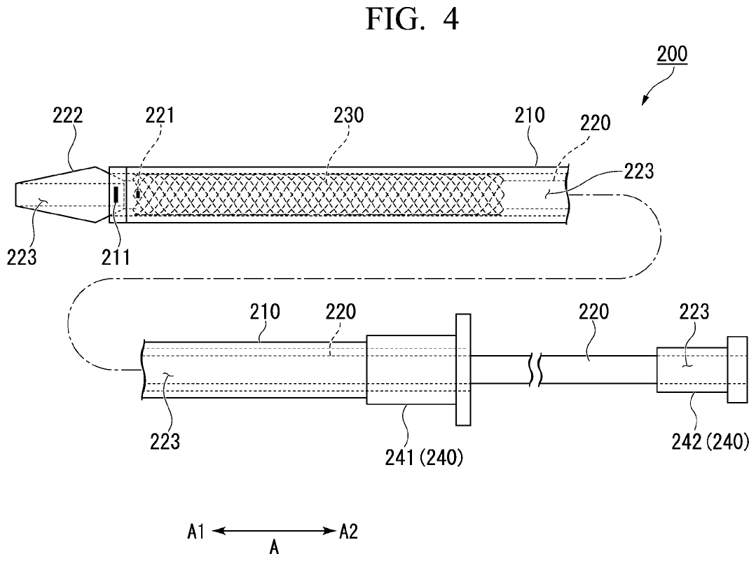

FIG. 4 shows a stent delivery device 200.

The stent delivery device 200 is elongated as a whole and includes an outer tubular member 210, an inner tubular member 220, a stent 230, and an operating portion 240.

The outer cylindrical member 210 is formed of resin or the like in a cylindrical shape and has flexibility. The outer cylinder member 210 can be inserted through the channel tube 171 of the endoscope 100. An outer tube marker 211 that is an X-ray opaque metal marker is provided at the distal end of the outer tube member 210.

The inner cylinder member 220 has an outer diameter smaller than the inner diameter of the outer cylinder member 210 and can be passed through the internal space (lumen) of the outer cylinder member 210. The inner cylindrical member 220 is made of resin or the like and has flexibility. A distal end 222 having an outer diameter larger than that of the outer cylindrical member 210 is provided at the distal end of the inner cylindrical member 220.

The stent 230 is a tubular self-expanding stent and is formed by weaving wires. The stent 230 is accommodated in the gap between the outer tubular member 210 and the inner tubular member 220 in a state in which the inner tubular member 220 is passed through the inner tubular member 220 and the diameter of the stent 230 is reduced. The stent 230 is locked by a locking portion 221 formed on the outer peripheral surface of the inner tubular member 220. As a result, the stent 230 is positioned relative to the inner cylinder member 220 in a reduced diameter state, and does not move in the longitudinal direction A relative to the inner cylinder member 220.

The wire forming the stent 230 is a superelastic alloy, the main material of which is NiTi. A superelastic alloy composed mainly of NiTi is not permanently deformed w % ben it is woven, and the woven shape is memorized by applying a heat treatment in a woven state. The stent 230 may be a laser-cut type stent formed by cutting a metal tube with a laser.

The operating portion 240 is provided on the proximal end side A2 of the outer cylinder member 210 and the inner cylinder member 220, and is capable of moving the outer cylinder member 210 relative to the inner cylinder member 220 in the longitudinal direction A. The operating portion 240 has an outer cylinder operating portion 241 that drives the outer cylinder member 210 and an inner cylinder operating portion 242 that drives the inner cylinder member 220.

The operator can place the stent 230 by exposing the accommodated stent 230 by moving the outer tube operating portion 241 to the proximal side A2 with respect to the inner tube operating portion 242. The operator can also recapture the stent 230 by moving the outer tube operating portion 241 toward the distal end side A1 with respect to the inner tube operating portion 242.

A guide wire lumen 223 through which the guide wire GW is inserted is formed from the distal end 222 to the inner cylinder operating portion 242 of the operating portion 240 via the inner cylinder member 220.

### [Driving Device 300]

FIG. 5 is a functional block diagram of the driving device 300.

The driving device 300 includes a driving device main body 310, a delivery device driving device 370, and a connecting portion driving device 380. The driving device main body 310 and the delivery device driving device 370 may be an integrated device. Further, the driving device main body 310 and the connecting portion driving device 380 may be an integrated device.

The drive device main body 310 has an adapter 320, an operation reception portion 330, an air supply/suction driving portion 340, a wire driving portion 350, and a drive controller 360.

The adapter 320 has an operation device adapter 321 and an endoscope adapter 322. The operation device adapter 321 is an adapter to which the operation cable 601 is detachably connected. The endoscope adapter 322 is an adapter to which the first detachable portion 1501 of the endoscope 100 is detachably connected.

The operation reception portion 330 receives operation input from the operation device 600 via the operation cable 601. When the operation device 600 and the drive device 300 communicate with each other not by wired communication but by wireless communication, the operation reception portion 330 has a known wireless reception module.

The air supply/suction driving portion 340 is connected to the air supply/suction tube 172 inserted into the internal path 101 of the endoscope 100. The air supply/suction driving portion 340 includes a pump or the like, and supplies air to the air supply/suction tube 172. Also, the air supply/suction driving portion 340 sucks air from the air supply/suction tube 172.

The wire driving portion 350 includes a drive mechanism having a motor and the like, and drives the bending wires (operation wires and elevator operation wires) 160. Specifically, the wire driving portion 350 includes a bending driving portion that drives the operation wire to bend the bending portion 112, and an elevator driving portion that drives the elevator operating wire to drive the elevator. The wire driving portion 350 has an encoder that detects the amount of traction of the bending wire 160. The detected amount of traction is acquired by drive controller 360.

The drive controller 360 controls the drive device 300 as a whole. The drive controller 360 acquires the operation input received by the operation reception portion 330. The drive controller 360 controls the air supply/suction driving portion 340, the wire driving portion 350, the connecting portion drive device 380, and the delivery device drive device 370 based on the acquired operation input and the like.

The drive controller 360 includes a processor 361, a memory 362, a storage portion 363 capable of storing programs and data, and an input/output control portion 364. The drive controller 360 is a programmable computer. The functions of the drive controller 360 are implemented by the processor 361 executing programs. At least some functions of the drive controller 360 may be realized by dedicated logic circuits.

The input/output control portion 364 is connected to the operation reception portion 330, the air supply/suction driving portion 340, the delivery device driving device 370, the connecting portion driving portion 380, the video control portion 400, the input device (not shown), and the network equipment (not shown). Under the control of the processor 361, the input/output control portion 364 transmits and receives data and control signals to and from connected devices.

The drive controller 360 may further have components other than the processor 361, the memory 362, the storage portion 363 and input/output control portion 364. For example, the drive controller 360 may further include an image calculation portion that performs part or all of the image processing and image recognition processing. By further having an image calculation portion, the drive controller 36) can execute specific image processing and image recognition processing at high speed. The image calculation portion may be mounted in a separate hardware device connected via a communication line.

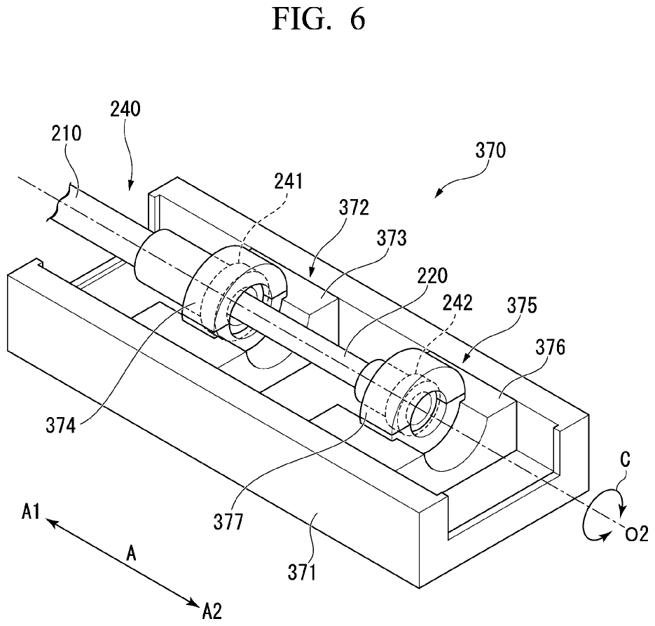

FIG. 6 is a diagram showing the delivery device driving device 370.

The delivery device driving device 370 is a device to which the operating portion 240 of the stent delivery device 200 is detachably connected. The delivery device driving device 370 can operate the connected operating portion 240 based on instructions from the driving controller 360.

The delivery device driving device 370 has a main body 371, an outer cylinder driving portion 372 and an inner cylinder driving portion 375.

The outer cylinder driving portion 372 is detachably fixed to the outer cylinder operating portion 241. The outer cylinder driving portion 372 has an outer cylinder forward/backward driving portion 373 and an outer cylinder rotating driving portion 374. The outer cylinder advancing/retracting driving portion 373 is driven by a motor or the like, and moves the outer cylinder operating portion 241 forward/backward in the longitudinal direction A with respect to the main body 371. The outer cylinder rotation driving portion 374 is driven by a motor or the like, and rotates the outer cylinder operating portion 241 about the central axis O2 in the longitudinal direction A with respect to the outer cylinder advancing/retracting driving portion 373.

The inner cylinder driving portion 375 is detachably fixed to the inner cylinder operating portion 242. The inner cylinder driving portion 375 has an inner cylinder advancing/retracting driving portion 376 and an inner cylinder rotating driving portion 377. The inner cylinder advancing/retracting driving portion 376 is driven by a motor or the like, and moves the inner cylinder operating portion 242 forward/backward in the longitudinal direction A with respect to the main body 371. The inner cylinder rotation driving portion 377 is driven by a motor or the like, and rotates the inner cylinder operating portion 242 about the central axis O2 in the longitudinal direction A with respect to the inner cylinder forward/backward driving portion 376.

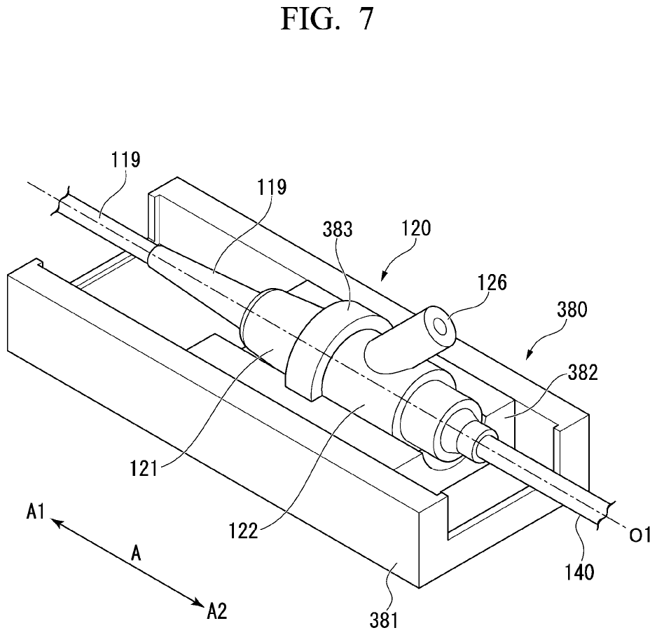

FIG. 7 is a diagram showing the connecting portion driving device 380.

The connecting portion driving device 380 is a device to which the connecting portion 120 of the endoscope 100 is detachably connected. The connecting portion drive device 380 can operate the connected connecting portion 120 based on instructions from the drive controller 360.

The connecting portion driving device 380 has a main body 381, an advancing/retracting driving portion 382 and a roll rotation driving portion 383.

The advancing/retracting driving portion 382 is detachably fixed to the connecting portion main body 122 of the connecting portion 120. The advancing/retracting driving portion 382 is driven by a motor or the like, and advances/retracts the connecting portion main body 122 in the longitudinal direction A with respect to the main body 381. The drive controller 36) can advance and retract the insertion portion 110 of the endoscope 100 in the longitudinal direction A by driving the advancing/retracting driving portion 382 to advance and retract the connecting portion main body 122 in the longitudinal direction A.

The cylindrical member 121 of the connecting portion 120 is detachably fixed to the roll rotation driving portion 383. The roll rotation driving portion 383 is driven by a motor or the like, and rotates the cylindrical member 121 about the central axis O1 in the longitudinal direction A with respect to the connecting portion main body 122 fixed to the advancing/retracting driving portion 382. The drive controller 360 drives the roll rotation driving portion 383 to rotate the cylindrical member 121 with respect to the connecting portion main body 122, thereby rotating the insertion portion 110 of the endoscope 100 in the longitudinal direction A in a roll rotation RO (FIG. 2).

### [Video Control Device 400]

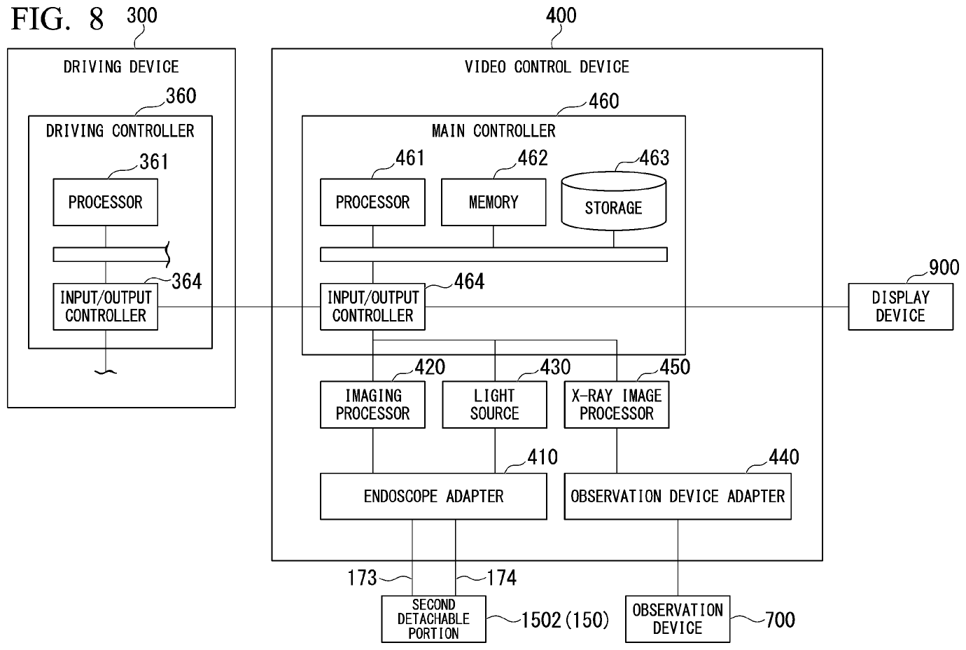

FIG. 8 is a functional block diagram of the video control device 400.

The video control device 400 includes an endoscope adapter 410, an imaging processing portion 420, a light source portion 430, an observation device adapter 440, an observation image processing portion 450, and a main controller 460.

The endoscope adapter 410 is an adapter to which the second detachable portion 1502 of the endoscope 100 is detachably connected.

The imaging processing portion 420 converts the imaging signal acquired from the imaging portion 111c of the distal end portion 111 via the imaging cable 173 into a captured image.

The light source portion 430 generates illumination light that irradiates the object to be imaged. The illumination light generated by the light source portion 430 is guided to the illumination portion 111b of the distal end portion 111 via the light guide 174.

The observation device adapter 440 is an adapter to which a connection cable (not shown) connected to the observation device 700 is detachably connected.

The observation image processing portion 450 acquires the X-ray observation signal from the observation device 700 via the connection cable. The observation image processing portion 450 converts the acquired X-ray observation signal into an X-ray observation image. When the observation device 700 includes a device for observing a patient by CT, the observation image processing portion 450 converts the obtained CT observation signal into a CT image. When the observation device 700 includes a device for observing a patient by MRI, the observation image processing portion 450 converts the acquired MRI observation signal into an MRI image. Note that the observation device adapter 440 and the observation image processing portion 450 may be devices separated from the video control device 400.

The main controller 460 has a processor 461, a program-readable memory 462, a storage portion 463, and an input/output control portion 464. The main controller 460 is a computer capable of executing programs. The functions of the main controller 460 are implemented by the processor 461 executing programs. At least part of the functions of the main controller 460 may be realized by a dedicated logic circuit.

The storage portion 463 is a non-volatile recording medium that stores the above-described programs and necessary data. The storage portion 463 is composed of, for example, a ROM, a hard disk, or the like. A program recorded in the storage portion 463 is read into the memory 462 and executed by the processor 461.

The input/output control portion 464 is connected to the imaging processing portion 420, the light source portion 430, the observation image processing portion 450, the driving device 300, the display device 900, the input device (not shown), and the network equipment (not shown). Under the control of the processor 461, the input/output control portion 464 transmits and receives data and control signals to and from connected devices.

The main controller 460 can perform image processing on the captured image acquired by the imaging processing portion 420 and the X-ray observation image acquired by the observation image processing portion 450. The main controller 460 can generate GUI images and CG images for the purpose of providing information to the operator. The main controller 460 can display captured images. X-ray observation images, GUI images, and CG images on the display device 900.

The main controller 460 is not limited to an integrated hardware device. For example, the main controller 460 may be configured by separating a part of it as a separate hardware device and then connecting the separated hardware device with a communication line. For example, the main controller 460 may be a cloud system that connects the separated storage portions 463 with a communication line.

The main controller 460 may further have components other than the processor 461, memory 462, storage portion 463, and input/output control portion 464. For example, the main controller 460 may further have an image calculation portion that performs some or all of the image processing and image recognition processing. By further having an image calculation portion, the main controller 460 can execute specific image processing and image recognition processing at high speed. The image calculation portion may be mounted in a separate hardware device connected via a communication line.

### [Operation of Endoscope System 1000]

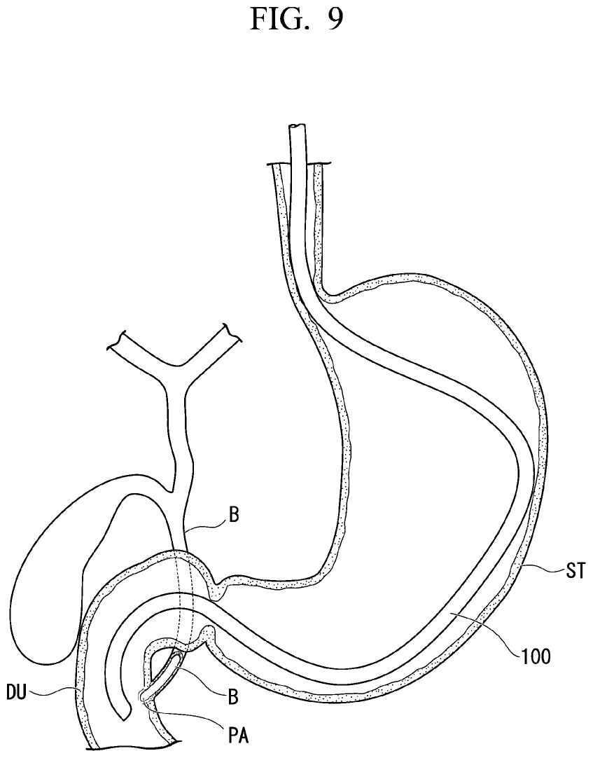

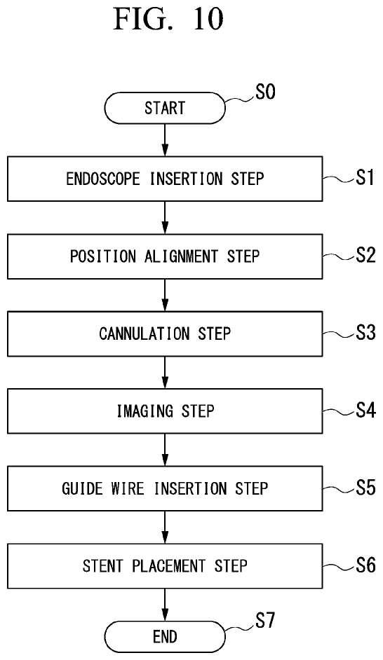

Next, the operation of the endoscope system 1000 according to this embodiment will be described. Specifically, a procedure for placing the stent 230 in the bile duct B by endoscopic retrograde cholangiopancreatography (ERCP) will be described. FIG. 9 shows a bile duct B in which a stent 230 is placed. FIG. 10 is a flow chart showing the steps of the procedure.

### <Step S1. Endoscope Insertion Step>

In step S1, the operator inserts the insertion portion 110 of the endoscope 100 into the patient's lumen through a natural opening such as the mouth. The operator can bend the bending portion 112 by operating the operation device 600. The operator inserts the distal end portion 1l1 of the endoscope 100 into the duodenum DU. The operator may insert the insertion portion 110 of the endoscope 100 into the patient's lumen using an overtube. The endoscope 100 may be manually inserted by an operator's hand, or may be inserted by electric drive.

### <Step S2: Position Alignment Step>

In step S2, the operator adjusts the position of the distal end portion 111 of the endoscope 100 so that the papilla PA is within the imaging range of the imaging portion 111c of the endoscope 100. The operator may manually align the endoscope 100 (manual mode). Further, the operator may automatically perform positioning of the endoscope 100 by the control device 500 (auto mode), as described below.

### <Step S2: Alignment Step (Auto Mode)>

When the control device 500 automatically aligns the endoscope 100, the main controller 460 (mainly the processor 461) drives the wire driving portion 350 by communicating with the drive controller 360 to bend the bending portion 112 of the insertion portion 110 vertically and horizontally. In addition, the main controller 460 drives the connecting portion driving device 380 by communicating with the drive controller 360 to move the insertion portion 110 back and forth in the longitudinal direction A and roll. The main controller 460 drives the bending portion 112 and the connecting portion driving device 380 based on the captured image acquired from the imaging portion 111c so that the papilla PA is within the imaging range. At this time, the main controller 460 may use an observation image (such as an X-ray image) acquired from the observation device 700.

### <Step S3: Cannulation Step>

The operator inserts a cannula from the papilla PA into the bile duct B in step S3. Specifically, a cannula is inserted into the channel tube 171 of the endoscope 100 to protrude from the distal end opening 116, and the cannula is inserted into the bile duct B.

### <Step S4: Imaging Step>

In step S4, the operator injects the contrast medium into the cannula to flow the contrast medium into the bile duct B through the cannula. The operator obtains an X-ray image showing the bile duct B and the like by performing X-ray imaging using the observation device 700. The operator can acquire CT images and MRI images.

### <Step S5: Guide Wire Insertion Step>

The operator inserts the guide wire GW into the cannula, protrudes the guide wire GW from the cannula, and inserts the guide wire GW into the bile duct B in step S5. Next, the operator withdraws the cannula while leaving the guide wire GW in the bile duct B. Thereby, only the guide wire GW is left in the bile duct B.

### <Step S6: Stent Placement Step>

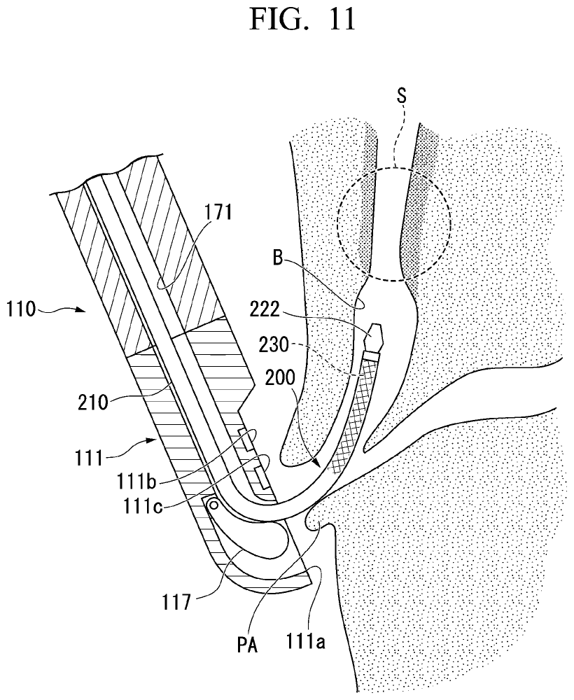

FIG. 11 is a diagram showing the stent delivery device 200 moved to the vicinity of the stenosis S. The operator inserts the stent delivery device 200 into the bile duct B along the guide wire GW in step S6. The operator manipulates the stent delivery device 200 under X-ray fluoroscopy to move the stent delivery device 200 along the guide wire GW to the liver side, and moves the portion containing the stent 230 in the stent delivery device 200 to the vicinity of the stenosis S. The control device 500 assists the stent placement step (step S6) as described below.

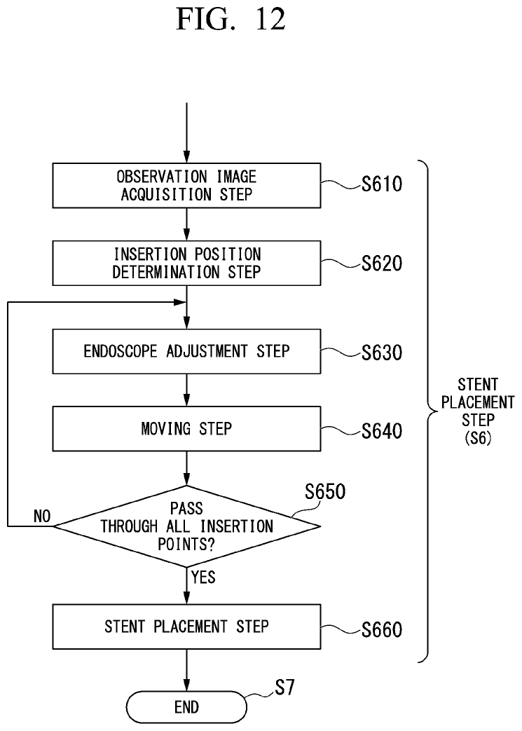

Hereinafter, description will be made along the control flowchart of the main controller 460 of the control device 500 in the stent placement step (step S6) shown in FIG. 12. When the operator inputs an operation start instruction to the control device 500, the main controller 460 performs initialization and then starts the following control. First, the main controller 460 (mainly processor 461) executes step S610.

### <Step S610: Observation Image Acquisition Step>

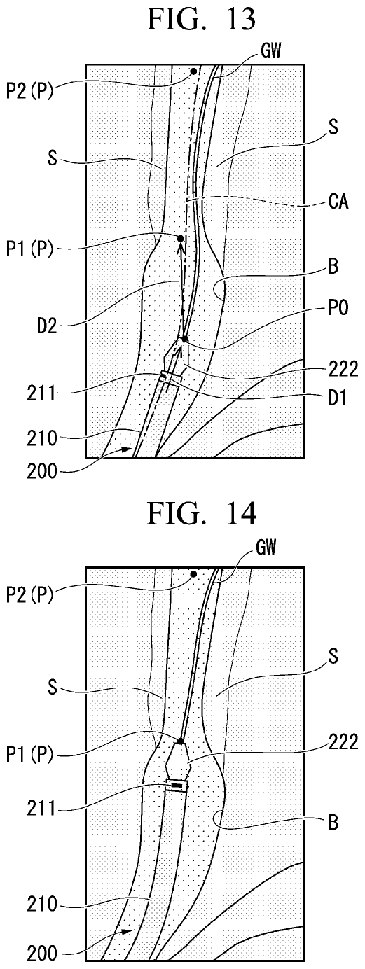

FIG. 13 is an X-ray image acquired by the observation image acquisition process.

In step S610, the main controller 460 acquires an X-ray image (observation image) showing the bile duct B and the like. The main controller 460 can acquire CT images and MRI images. The main controller 460 then executes step S620.

### <Step S620: Insertion Position Determination Step>

The main controller 460 determines the insertion position P of the stent delivery device 200 to be inserted through the stenosis S based on the observation image in step S620. The insertion position P refers to a position or coordinates through which the endoscope system 1000 recommends that the stent delivery device 200 pass. The stenosis S shown in FIG. 13 is not uniformly constricted against the wall surface of the bile duct B, and has an asymmetrical shape with respect to the central axis CA of the bile duct B. Therefore, the stent delivery device 200 tends to get caught in the stenosis S when advanced toward the stenosis S along the central axis CA. If the stent delivery device 200 is caught in the stenosis S, it is difficult for the distal end 222 of the stent delivery device 200 to pass through the stenosis S and enter the liver side. Therefore, the main controller 460 determines the insertion position P of the stent delivery device 200 such that the stent delivery device 200 does not come into contact with the stenosis S based on the observation image.

The main controller 460 recognizes the “current position P0” of the distal end 222 from, for example, the outer cylinder marker 211 appearing in the observation image. Next, as shown in FIG. 13, the main controller 460 determines a position that is as far away from the wall surface of the stenosis S as possible in the radial direction of the bile duct B and has a low possibility of coming into contact with the wall surface of the stenosis S as the “insertion position P”. The insertion position P includes a “final insertion position PL” where the distal end 222 of the stent delivery device 200 passes through the stenosis S and is finally positioned.

The insertion position P may include, in addition to the final insertion position PL, an “intermediate insertion position” set in the insertion path from the current position P0 of the distal end 222 of the stent delivery device 200 to the final insertion position PL. The insertion position P shown in FIG. 13 includes a first intermediate insertion position P1 and a second intermediate insertion position P2 on the path from the current position P0 to the final insertion position PL. The main controller 460 can control the insertion path of the stent delivery device 200 more precisely as the number of intermediate insertion positions to be set increases.

The main controller 460 may determine the insertion position P from a three-dimensional model of the bile duct B created based on multiple observation images observed from different angles. By using the three-dimensional model of the bile duct B, the main controller 460 can determine the insertion position P more accurately. In this case, the insertion position determination step is a step of determining a recommended position and coordinates for the stent delivery device 200 to be inserted from the three-dimensional model of the bile duct B. The insertion position determination step is a step of determining the recommended position and coordinates for insertion so that the distal end of the stent delivery device 2(x) does not contact the stenosis S from the three-dimensional model including the stenosis S of the bile duct B.

The main controller 460 may determine the insertion position P based on operator input from a three-dimensional model of the bile duct B created based on multiple observation images observed from different angles. The main controller 460 can set the insertion path in consideration of the bile duct shape, for example, by the operator specifying the insertion position P on the three-dimensional model of the bile duct B via an interface. Further, the insertion position P determined by the main controller 460 may be presented to the operator, and the operator may be allowed to finely adjust the insertion position P via an interface.

After determining at least one insertion position P, the main controller 460 executes step S630.

### <Step S630: Endoscope Adjustment Step>

In step S630, the main controller 460 controls the driving device 300 based on the observation image to drive the endoscope 100, and adjusts the position of the stent delivery device 200 so that the stent delivery device 200 passes through the insertion position P. Specifically, the driving device 300 drives at least one of the wire driving portion 350 (curving driving portion, elevator driving portion) and the connecting portion driving portion 380 (advancing/retracting driving portion 382, roll rotation driving portion 383), to adjust the position of the stent delivery device 200.

Based on the observed image, the main controller 460 adjusts the position of stent delivery device 200, so that the first direction D1 in which the distal end 222 of the stent delivery device 200 faces substantially coincides with the second direction D2 from the current position P0 of the distal end 222 of the stent delivery device 200 toward the closest insertion position P (first intermediate insertion position P1 in the case of FIG. 13). It is not necessary for the first direction D1 completely coincident with the second direction if the stent delivery device 200 can pass through stenosis S.

The first direction D1, which is adjusted to substantially match the second direction D2, may be the advancing/retracting direction of the stent delivery device 200 regulated by the elevator 117. The main controller 460 drives the elevator driving portion of the wire driving portion 350 to move the elevator 117 so that the first direction D1 substantially coincides with the second direction D2. It is not necessary for the first direction D1 completely coincident with the second direction if the stent delivery device 200 can pass through stenosis S.

The main controller 460 may calculate the first direction D1 and the second direction D2 from a three-dimensional model of the bile duct B created based on multiple observation images observed from different angles. By using the three-dimensional model of bile duct B, the main controller 460 can more accurately adjust the position of stent delivery device 200.

The main controller 460 drives the endoscope 100 to adjust the first direction D1 in which the distal end 222 of the stent delivery device 200 faces, and then executes step S640.

### <Step S640: Moving Step>

The main controller 460 moves the stent 230 to the insertion position P by driving the delivery device driving device 370 and operating the operating portion 240 of the stent delivery device 200 in step S640. The operator may remove the operating portion 240 of the stent delivery device 200 from the delivery device driving device 370 and manually operate the operating portion 240 of the stent delivery device 200.

FIG. 14 shows the stent delivery device 200 moved to the first intermediate insertion position P1. The main controller 460 drives the delivery device drive device 370 by communicating with the drive controller 360 to operate the operating portion 240 of the stent delivery device 200. Specifically, the main controller 460 advances and retracts the stent delivery device 200 in the longitudinal direction A until the distal end 222 of the stent delivery device 200 substantially coincides with the first intermediate insertion position P1. The main controller 460 advances and retracts the entire stent delivery device 200 by driving the delivery device driving device 370 to simultaneously advance and retract the outer cylinder driving portion 372 and the inner cylinder driving portion 375. The main controller 460 then executes step S650.

### <Step S650: Final Insertion Position Determination Step>

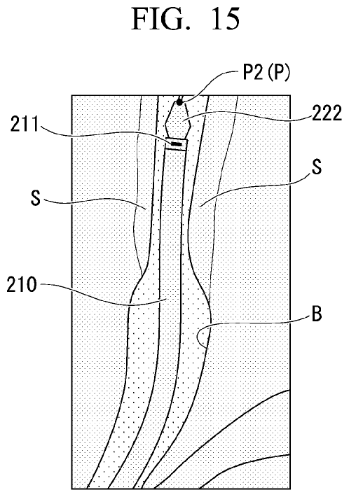

FIG. 15 is a diagram showing the stent delivery device 200 moved to the second intermediate insertion position P2. The main controller 460 determines in step S650 whether the current position P0 of the distal end 222 of the stent delivery device 200 is the final insertion position PL. If the current position P0 is not the final insertion position PL, the main controller 460 re-performs step S630 (observation data acquisition step endoscope adjustment step) and step S640 (movement step) to is moved to the second intermediate insertion position P2, which is the next insertion position P.

When the current position P0 of the distal end 222 of the stent delivery device 200 is the final insertion position PL, the main controller 460 executes step S660.

### <Step S660: Stent Placement Step>

In step S660, the main controller 460 drives the delivery device driving device 370 to operate the operating portion 240 of the stent delivery device 200 to automatically place the stent 230. The delivery device driving device 370 exposes the accommodated stent 230 by moving the outer tube operating portion 241 to the proximal end side A2 with respect to the inner tube operating portion 242, and places the stent 230 therein. The operator may remove the operating portion 240 of the stent delivery device 200 from the delivery device driving device 370 and manually operate the operating portion 240 of the stent delivery device 200.

After placing the stent 230, the operator pulls out the stent delivery device 200 excluding the stent 230 from the body.

A part or all of the control flowchart of the main controller 460 described above may be implemented by the drive controller 360.

According to the endoscope system 1000 of this embodiment, it is easy to place the stent 230 at the target position. Even if the stenosis S is not uniformly narrowed with respect to the wall surface of the bile duct B and has an asymmetrical shape with respect to the central axis CA of the bile duct B, the endoscope system 1000 can contact the wall surface of the stenosis S. A position with low flexibility is determined as an “insertion position P”, and the first direction D1 in which the stent delivery device 200 faces can be adjusted by driving the endoscope 100 or the like.

As described above, the first embodiment has been described in detail with reference to the drawings, but the specific configuration is not limited to this embodiment, and design changes and the like are included within the scope of the present invention. In addition, the constituent elements shown in the above-described first embodiment and modifications shown below can be combined as appropriate.

### Second Embodiment

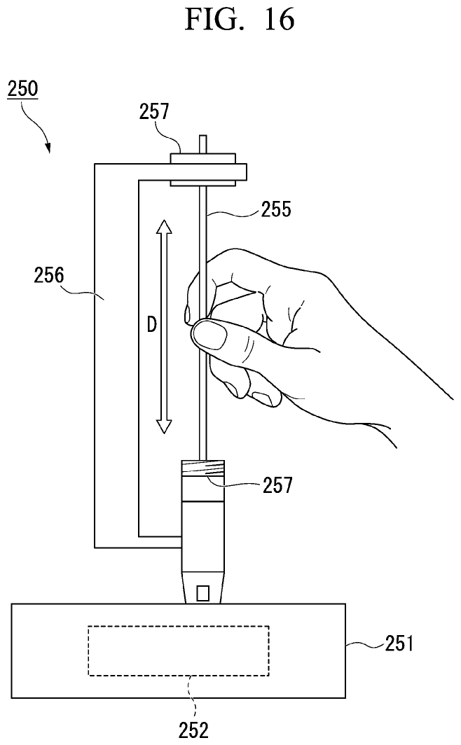

A second embodiment will be described with reference to FIG. 16. The endoscope system according to the second embodiment differs from the endoscope system 1000 according to the first embodiment in that it further includes a second input device 250. In the following description, the same reference numerals are given to the same configurations as those already described, and redundant descriptions will be omitted.

FIG. 16 is a diagram showing the second input device 250.

The second input device 250 is an operation device that remotely operates treatment tools such as the stent delivery device 200. The second input device 250 is connected to the driving device 300 by wireless communication, for example. The drive device 300 receives an operation input signal from the second input device 250 and drives the treatment instrument based on the operation input signal. The second input device 250 may be wire-connected to the drive device 300 by a cable, a connector, or the like.

The second input device 250 includes a base 251, a controller 252, an operation sheath 255, a support arm 256, and sheath holding portions 257.

The controller 252 is provided on the base 251 and connected to the operating sheath 255. The controller 252 has a sensor that detects a sliding operation (advancing and retracting operation) with respect to the operating sheath 255 and a driving portion that drives the operating sheath 255.

The operating sheath 255 is a sheath similar to the sheath of the treatment instrument. The operating sheath 255 may have a similar feel and texture to the sheath of the treatment instrument.

The support arm 256 is a member that linearly supports the operation sheath 255. The support arm 256 is U-shaped and has one end fixed to the base 251.

The sheath holding portions 257 are provided at both ends of the support arm 256 and support the operation sheath 255 so as to be able to advance and retract along the longitudinal direction D of the operation sheath 255.

Next, the action of the second input device 250 will be described.

The operator can remotely operate the stent delivery device 200 using the second input device 250 in step S640 (moving step). When the operator inputs a slide operation for sliding the operation sheath 255 along the longitudinal direction D, the controller 252 transmits the detected slide operation to the driving device 300.

The drive controller 360 of the driving device 300 drives the delivery device driving device 370 based on the received slide operation, and advances and retracts the entire stent delivery device 200 by simultaneously advancing and retracting the outer cylinder driving portion 372 and the inner cylinder driving portion 375.

For example, when the operator slides the operating sheath 255 toward the base 251, the delivery device driving device 370 advances the stent delivery device 200 as a whole. Also, when the operator slides the operating sheath 255 away from the base 251, the delivery device driving device 370 moves the entire stent delivery device 200 backward.

The controller 252 can provide tactile feedback to the operator by driving the operating sheath 255 with the driving portion. For example, the controller 252 uses the driving portion to apply a force to the operating sheath 255 in a direction opposite to the direction in which the operating sheath 255 advances and retracts, so that it is possible to feed back to the operator the “resistance” that occurs when the operating sheath 255 is advanced and retracted. In addition, the controller 252 can feed back a “warning” from the control device 500 to the operator by applying vibration to the operating sheath 255 using the driving portion.

When the main controller 460 determines in step S640 that the distal end 222 of the stent delivery device 200 is likely to collide with the wall surface of the stenosis S, for example, the main controller 460 feeds back a “warning” to the operator by applying vibration to the operating sheath 255.

When the main controller 460 determines in step S640 that the trajectory of the distal end 222 of the stent delivery device 200 deviates greatly from the insertion path determined in step S2, the main controller 460 feeds back a “warning” to the operator by applying vibration to the operating sheath 255. For example, the main controller 460 determines that the distal end 222 is greatly deviated from the insertion path when the distal end 222 protrudes from a range of about 80% from the center of the path cross section.

According to the endoscope system of this embodiment, it is easy to place the stent 230 at the target position. The endoscope system of the present embodiment can enhance the safety of a procedure by providing tactile feedback of a warning to the operator.

As described above, the second embodiment has been described in detail with reference to the drawings, but the specific configuration is not limited to this embodiment, and design changes and the like are also included within the scope of the present invention. Also, the constituent elements shown in the second embodiment described above and the modifications shown below can be combined as appropriate.

Although the treatment tool in the above embodiment is the stent delivery device 200, the treatment tool used in the endoscope system is not limited to this. A treatment instrument used in the endoscope system may be a balloon, a bucket, biopsy forceps, a guide wire, or the like.

In the above embodiment, the tactile feedback is provided by the second input device 250, but the device that provides tactile feedback is not limited to this. The instrument that provides tactile feedback may be a wearable device.

What is claimed is:

1. A stent delivery system, comprising:
a stent delivery device configured to carry a stent to a stenosis and indwell the stent;
an observation device configured to observe the stenosis; and
one or more processors comprising hardware, the one or more processors being configured to:
acquire an observation image from the observation device, and
determine, based on the observation image, at least one insertion position, which is a position within the stenosis where the stent delivery device is recommended to be inserted.

2. The stent delivery system according to claim 1, wherein the one or more processors being configured to determine the at least one insertion position from a three-dimensional model created based on a plurality of observation images, each observed from a different angle.

3. The stent delivery system according to claim 1, further comprising:
an endoscope having an insertion portion formed with a channel through which the stent delivery device is inserted; and
a driving actuator configured to drive the endoscope,
wherein the one or more processors are configured to:
control the driving actuator based on the observation image to drive the endoscope, and
adjust a position of the stent delivery device so that the stent delivery device passes through the at least one insertion position.

4. The stent delivery system according to claim 3, wherein the driving actuator comprises:
a bending driving actuator configured to bend the insertion portion;
an elevator driving actuator configured to drive an elevator provided in the channel of the insertion portion;
a forward/backward driving actuator configured to move the insertion portion back and forth; and
a roll rotation driving actuator configured to rotate the insertion portion in a roll,
wherein the driving actuator adjusts the position of the stent delivery device by driving at least one of the bending driving actuator, the elevator driving actuator, the forward/backward driving actuator, and the roll rotation driving actuator.

5. The stent delivery system according to claim 3, wherein the one or more processors are configured to adjust the position of the stent delivery device, based on the observation image, such that a first direction in which a distal end of the stent delivery device faces substantially coincides with a second direction from the distal end of the stent delivery device toward the at least one insertion position.

6. The stent delivery system according to claim 5, wherein the one or more processors being configured to calculate the first direction and the second direction from a three-dimensional model created based on a plurality of observation images, each observed from a different angle.

7. The stent delivery system according to claim 1, further comprising a driving actuator configured to drive the stent delivery device,
wherein the one or more processors being configured to control the driving actuator to drive the stent delivery device, and move the stent delivery device so that the stent delivery device passes the at least one insertion position.

8. A stent delivery method for inserting a stent delivery device to a stenosis position, the method comprising:
obtaining an observation image from an observation device used to observe a stenosis;
determining at least one insertion position, which is a position in a stenosis where the stent delivery device is recommended to be inserted, based on the observation image.

9. The stent delivery method according to claim 8, wherein the determining comprises determining the at least one insertion position from a three-dimensional model created based on a plurality of observation images, each observed from a different angle.

10. The stent delivery method according to claim 8, further comprising:
adjusting the position of the stent delivery device inserted through a channel formed in the insertion portion of an electrically driven endoscope so that the stent delivery device passes through the at least one insertion position based on the observation image.

11. The stent delivery method according to claim 10, wherein, the adjusting comprises adjusting the position of the stent delivery device, based on the observation image, so that a first direction in which a distal end of the stent delivery device faces substantially coincides with a second direction from the distal end of the stent delivery device toward the at least one insertion position.

12. The stent delivery method according to claim 11, wherein the adjusting comprises calculating the first direction and the second direction from a three-dimensional model created based on a plurality of observation images, each observed from a different angle.

13. The stent delivery method according to claim 8, further comprising driving the stent delivery device to move the stent delivery device so that the stent delivery device passes through the at least one insertion position.

14. A control device for a stent delivery system, the stent delivery system including a stent delivery device that carries and indwells a stent to a stenosis, an observation device used to observe the stenosis, and one or more processors comprising hardware,
wherein the one or more processors being configured to determine at least one insertion position, which is a position within the stenosis where the stent delivery system is recommended to be inserted, based on the observation image acquired from the observation device.

15. The control device according to claim 14, wherein the one or more processors being configured to determine the at least one insertion position from a three-dimensional model created based on a plurality of observation images, each observed from a different angle.

16. The control device according to claim 14, wherein
an endoscope comprising an insertion portion formed with a channel through which the stent delivery device is inserted, and
a driving actuator for driving the electric endoscope, and
one or more processors being configured to control the driving actuator based on the observation image to drive the endoscope, and adjust the position of the stent delivery device so that the stent delivery device passes through the at least one insertion position.

17. The control device according to claim 16, wherein the one or more processors being configured to adjust the position of the stent delivery device, based on the observation image, such that a first direction in which the distal end of the stent delivery device faces substantially coincides with a second direction from the distal end of the stent delivery device toward the at least one insertion position.

18. The control device according to claim 17, wherein the one or more processors are configured to calculate the first direction and the second direction from a three-dimensional model created based on a plurality of observation images, each observed from a different angle.

19. The control device according to claim 14, wherein the one or more processors being configured to:
control a driving actuator that drives the stent delivery device, and
control the driving actuator to drive the stent delivery device, and move the stent delivery device so that the stent delivery device passes the at least one insertion position.
